(19) United States
(12) Patent Application Publication
(10) Pub. No.: US 2004/0142039 A1
(43) Pub. Date: Jul. 22, 2004

(54) Solid and semi-solid polymeric ionic conjugates

(75) Inventors: Shalaby W. Shalaby, Anderson, SC (US); Jaymin C. Shah, Waterford, CT (US); Joel T. Corbett, Seneca, SC (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/693,383

(22) Filed: Oct. 24, 2003

Related U.S. Application Data
(60) Provisional application No. 60/422,832, filed on Oct. 31, 2002.

(51) Int. Cl. A61K 31/519; A61K 9/14

(52) U.S. Cl. 424/486; 514/259.41

(57) ABSTRACT

Aqueous solubility of drugs including insoluble or poorly soluble drugs such as ziprasidone is improved using a functional polymer to form an ionic conjugate with said drug.

This application claims priority under 35 USC 119(e) of U.S. Provisional 60/422,832, filed Oct. 31, 2002.

### FIELD OF THE INVENTION

The invention relates to improving the aqueous solubility of pharmaceutical compounds. In a particular aspect, the invention pertains to a solid or semi-solid ionic conjugate comprised of a pharmaceutical compound and a functional polymer.

BACKGROUND OF THE INVENTION

Organic pharmaceutical compounds having a molecular weight in excess of about 200 Da and a limited number of hydrophilic functionalities, e.g. polar groups, are typically insoluble or poorly soluble in aqueous media, i.e. aqueous media of the type found in or comparable to that in a biological environment. In almost all instances, this lack of solubility compromises the bioavailability of the compound, hence its therapeutic effectiveness. Moreover, the fate of the insoluble fraction of such a compound can not be predicted once in the body, raising concerns as to side effects due in whole or part to the uncontrolled residence time of the drug in living tissues.

Methods to try and increase the water solubility of insoluble and poorly soluble compounds have been developed. Examples are: (1) increasing the drug surface area through size diminution, e.g. by jet milling; (2) converting the drug, if basic, into a simple salt with a strong low-molecular weight acid, e.g. sulfuric, hydrochloric, acetic, methane sulfonic or tartaric acids; or (3) using a surface active agent or a complexing agent such as macrocyclic cage-type compound to increase solubility.

The problem of ameliorating the water solubility of a drug so as to incorporate same into a practicable formulation is further aggravated when the drug is also insoluble or poorly soluble in common organic solvents such as acetone, low molecular weight alcohols, hydrocarbons, ethers and chlorocarbons. In particular, this can impair efforts to make simple organic acid salts of the drug.

Ionic conjugation of large molecular weight organic acids is known in the art for decreasing, rather than increasing, the solubility of water soluble compounds. For instance, ionic conjugation with water-insoluble, carboxylic-bearing polyesters has been used to modulate the solubility of water-soluble basic peptides to render them practically water-insoluble and permit control of their release profile see e.g. U.S. Pat. Nos. 5,665,702; 5,821,221; 5,863,985; 6,204,256; and 6,221,958.

There is a recognized and on-going need for techniques to increase the aqueous solubility of pharmaceutical compounds, especially those that are water-insoluble or poorly soluble in water, so as to facilitate their incorporation into pharmaceutical formulations and/or improve their bioavailability subsequent to administration.

### SUMMARY OF THE INVENTION

The present invention improves the aqueous solubility of pharmaceutical compounds. In a particular practice, the invention pertains to improving the aqueous solubility of insoluble or poorly soluble drug substances. In one aspect, the invention pertains to a solid ionic conjugate comprising a pharmaceutical compound and a functional polymer. In one embodiment, the solid ionic conjugate of the invention has an aqueous solubility greater than that of the pharmaceutical compound. In another embodiment the pharmaceutical compound used in the solid ionic conjugate is by itself insoluble or poorly soluble. The subject ionic conjugate imparts improved water solubility, enabling e.g. the otherwise insoluble or poorly soluble pharmaceutical compound to be incorporated into pharmaceutical formulations, including without limitation, controlled release, oral concentrate, injectable dosage forms and the like.

### DETAILED DESCRIPTION OF THE INVENTION

The invention relates to ionic conjugates of pharmaceutical compounds, preferably water insoluble or poorly soluble pharmaceutical compounds, (also referred to herein as “drug(s)” or “drug compound(s)”) with functional polymers such as e.g. carboxyl- or amine-bearing polyesters, copolyesters and/or copolyester-carbonates. The term “pharmaceutical compound(s)” as understood by the artisan, also includes organic compounds or substances that are drug candidates. The polymers are understood to be absorbable (biodegradable and pharmaceutically acceptable), hence suitable for pharmaceutical use. Also as used herein the term “solid” ionic conjugate includes conjugates that are semi-solid as well.

### Water Insoluble or Poorly Soluble Druci Compounds

The invention contemplates increasing the solubility of drug compounds. For example, the invention provides increased solubility in an aqueous environment. An aqueous environment in this regard can include tissue, blood and the like, as for example found at the site of mammalian administration for a drug, and/or include the aqueous environment associated with a given formulation or dosage form.

In one practice, it is preferred that the drug compounds are insoluble and poorly soluble. The terms “insoluble” and “poorly soluble” and related variations of same as used herein to characterize drug compounds in respect of their water solubility are readily understood by the artisan. For example, in one non-limiting embodiment, it is preferred if the drug has a water solubility of less than about 1 mg/ml, more preferably less than about 0.1 mg/ml.

Other drug compounds that may benefit from the present invention are those that are not soluble in common organic solvents. While the criteria of “not soluble in common organic solvents” is understood by the artisan, it is preferred that the drug in question in its free form be less than about 40% soluble (e.g. solubility of less than about 400 mg/ml), more preferably less that about 20% soluble, still more preferably less than about 10% soluble, and yet still more preferably less than about 5% soluble, in at least one of the following common organic solvents: acetone; low molecular weight alcohols, e.g. ethanol or isopropanol; hydrocarbons, e.g. toluene; ethers, e.g. diethyl ether; chlorocarbons, e.g. chloroform. In a separate embodiment, thus if the drug compound is “not soluble” in any one of the foregoing solvents, it can be used for ionic conjugation as herein contemplated. The drug compound can also be “not soluble” in more than one of the foregoing solvents and be used for the invention. Drug compounds contemplated for use in the invention can be natural or synthetic, acidic, or basic. When acidic, it is preferred that the counterpart functional polymer is basic; when basic, it is preferred that the counterpart functional polymer be acidic.

In another embodiment, the drug subject to ionic conjugation of the invention is an aryl-heterocyclic compound, particularly chosen from those having psychotropic effects, such as the chlorooxyindole class of such heterocyclics. Representative aryl-heterocyclic compounds for purposes of this invention are those described in U.S. Pat. No. 4,831,031 incorporated herein by reference. In a particular practice the drug in question is ziprasidone, i.e. 5-[2-[4-(1,2-benzisothiazol-3-yl)-1-piperazinyl]ethyl]-6-chloro-1,3-dihydro-2H-indol-2-one; while salt forms of ziprasidone may be used in the invention to the extent the polymer can form an ionic conjugate with same, it is preferred that the ziprasidone be in its free base form, which is known to be insoluble or poorly soluble in water.

### Functional Polymers

The functional polymers of the invention are those bearing moieties that provide suitable ionic attraction with the insoluble or poorly soluble drugs aforesaid to generate the ionic bonding whereby the conjugates of the invention form. Such moieties include those that render the polymer acidic, e.g. carboxyl groups; or basic, e.g. amine groups. Preferably, at least one such moiety is present per polymer chain molecule; more preferably, two such moieties, e.g. carboxyl groups, are present per polymer chain molecule. Without limitation, such polymers include carboxyl-bearing polyesters, copolyesters, polyalkylene carbonates and copolyester-carbonates; and amine-bearing polyesters, copolyesters, polyalkylene carbonates and copolyester-carbonates. It is preferred if the acidic or basic groups of the functional polymer are sufficiently accessible for purposes of forming the ionic conjugate, e.g. in the case of ziprasidone, that the acidic functional polymer have reasonably accessible carboxylic groups. The polymers of the invention are absorbable as stated above.

In one aspect, especially preferred for the conjugation of insoluble drugs, e.g. having a solubility of less than about 1 mg/ml, preferably less than about 0.1 mg/ml, in water, especially those that are basic, e.g. ziprasidone and the like, the functional polymers are preferably acidic, such as e.g. carboxyl-bearing polyesters and carboxyl-bearing copolyester carbonates that are made by ring-opening polymerization of one or more of the following cyclic polymers: lactide (L), glycolide (G), p-dioxanone (PD), ε-caprolactone (CL), 1,5-dioxepan-2-one (DOP), and trimethylene carbonate (TMC). The ring-opening polymerization occurs in the presence of a suitable acidic initiator, e.g. glycolic acid, lactic acid, citric acid, malic acid, tartaric acid, or mixtures thereof; and a suitable catalyst, such as an organometallic catalyst, preferably a transition metal based catalyst, e.g. stannous octanoate.

In another aspect, especially for those drugs that are acidic, e.g. sodium tenidap, the functional polymers are preferably basic, such as e.g. absorbable amine-bearing copolyesters or amine-bearing polyalkylene carbonates or amine-bearing copolyester carbonates that are made by ring-opening polymerization of one or more of the following cyclic polymers: lactide (L), glycolide (G), p-dioxanone (PD), ε-caprolactone (CL), 1,5-dioxepan-2-one (DOP), and trimethylene carbonate (TMC). The ring-opening polymerization occurs in the presence of a suitable basic initiator, preferably a hydroxylic basic initiator e.g. triethanolamine, N-hydroxyethyl piperazine, N-methyl-diethanolamine, N-diethyl-ethanolamine or mixtures thereof; and a suitable catalyst, such as an organometallic catalyst, preferably a transition metal based catalyst, e.g. stannous octanoate. In another practice of this aspect, the absorbable amine-bearing polyesters, polyalkylene carbonates and polyester carbonates described hereinbefore are used to form ionic conjugates with insoluble or poorly soluble drug compounds having a highly ionizable, pseudo-acid hydroxylic group, such as e.g. sodium tenidap.

In another aspect, carboxyl-bearing polypeptides, such as polyaspartic acid, are employed as the functional polymer to form ionic conjugates with a drug as hereinbefore described, said drug preferably basic.

In another aspect, a basic polypeptide, such as polylysine, is used to form ionic conjugates of drug compounds that have acid or pseudo-acid groups, such as e.g. sodium tenidap.

In another aspect, the functional polymer comprises a saccharide, including without limitation a cyclic oligosaccharide derivative with carboxyl groups on the outer surface and optionally a void cavity on the inner surface, which is typically hydrophobic. Examples of such a saccharide are cyclodextrins, especially those that have been functionalized to incorporate one or more carboxyl groups as hereinafter described. Cyclodextrins have the ability to form complexes with drug compounds such as ziprasidone as described in U.S. Pat. No. 6,232,304, incorporated herein by reference. For purposes of this invention, preferred cyclodextrins include without limitation: α-, β-, and γ-cyclodetxtrins, methylated cyclodextrins, hydroxypropyl-β-cyclodextrin (HPBCD), hydroxyethyl-β-cyclodextrin (HEBCD), branched cyclodextrins in which one or two glucoses or maltoses are enzymatically attached to the cyclodextrin ring, ethyl- and ethyl-carboxymethyl cyclodextrins, dihydropropyl cyclodextrins, and sulfoalkyl ether cyclodextrins, such as sulfobutyl ether-β-cyclodextrin (SBECD). The cyclodextrins can be unsubstituted or substituted in whole or in part as known in the art; mixtures of cyclodextrins can also be employed. Preferred cyclodextrins include γ-cyclodetxtrin, HPBCD, SBECD or mixtures thereof, SBECD being most preferred.

In one practice, the cyclodextrin is functionalized to include one or more carboxyl groups, which functionalized cyclodextrin is then effectively used as part of the functional polymer, the drug being ionically conjugated to the polymer units on the sugar (e.g. cyclodextrin). As an example of this cyclodextrin aspect, a basic insoluble drug as aforesaid is ionically conjugated with a carboxyl-bearing cyclodextrin water insoluble derivative, as described in e.g. U.S. Pat. Nos. 6,162,895 and 5,916,883, incorporated herein by reference, wherein the insoluble cyclodextrin derivative is made by a mixed partial acylation of cyclodextrin with a fatty acid anhydride and a cyclic anhydride; the mixed partial acylation results in a cyclodextrin bearing at least one unacylated hydroxylic group. This is followed by grafting the unacylated hydroxylic group of said cyclodextrin with one or more of the following cyclic monomers: lactide (L), glycolide (G), p-dioxanone (PD), ε-caprolactone (CL), 1,5-dioxepan-2-one (DOP), and trimethylene carbonate (TMC).

In another aspect, the functional polymer is an absorbable or non-absorbable acidic polymeric precursor wherein the polymeric chain of the precursor comprises one or more sulfonic groups. Such polymers are particularly useful for forming solid or semi-solid ionic conjugates with basic drugs.

### Ionic Conjugation

Representatively, the ionic conjugate of the invention may be made as follows: the drug as hereinbefore described is contacted with one or more functional polymers as described above under conditions effective to cause sufficient proton transfer whereby ionic conjugation between the basic aspects or moieties of said drug (or said polymer as the case may be) and said acidic aspects or moieties of said polymer (or the drug as the case may be) occurs. In one embodiment, effective conditions are provided by forming a solution of the drug and its functional polymer counterpart; for example, a solution of the ionic conjugate precursors, i.e. the drug compound and the functional polymer. The solution can be made using halocarbons such as a fluorocarbon, e.g. hexafluoro-isopropanol (HFIP) or trifluoroethanol and the like, as solvents. In another practice of this embodiment, the solvent (e.g. the halocarbon) is removed to provide a solid or semi-solid ionic polymeric conjugate without causing any substantial compromise to the stability of the conjugate; in another practice in this regard, the solvent is removed at or below room temperature, e.g. about 25° C., using, for example, reduced pressure. In yet another practice of this embodiment, the drug component (i.e. the basic, or acidic as the case may be, moieties of the drug component) of the dry solid or semi-solid conjugate are at least 30%, more preferably at least 60%, still more preferably at least 80% ionically conjugated to the acidic (or, respectively, basic) moieties of the polymer, and the resultant conjugate does not exhibit: (1) the melting point (Tₘ) of the original drug in a typical Differential Scanning Calorimetry (DSC) thermogram; or 2) crystalline reflections of a typical wide-angle X-ray diffraction pattern. The drug loadings in any given conjugate can be varied by percentages as would be understood in the art.

As used herein the term “mgA/ml” relates to the weight (in mg) of the pharmaceutical compound in its free form, e.g. ziprasidone free base, calculated per ml of composition in consideration. (For ziprasidone free base, molecular weight=412.9.)

### Pharmaceutical Formulations

Without limitation, the polymeric ionic conjugate of the invention is useful in a pharmaceutical formulation. The conjugates can be used e.g. to provide immediate release or controlled release injectable formulations and other dosage forms as herein described. The invention in a preferred aspect pertains to a controlled release formulation, such as a depot formulation, including without limitation injectable depot formulations, e.g. intramuscularly injectable depot formulations of ziprasidone. The formulations herein can be used to treat mammals, including humans, in need of treatment for illnesses including but not limited to schizophrenia and other psychotic disorders.

In one practice of the formulation aspect of the invention, the ionic conjugates are used with injectable, absorbable or biodegradable pharmaceutically acceptable vehicles to provide a controlled release effect. Controlled release includes, without limitation, the effect of modulating the release of the drug after administration to a mammal. For example, an absorbable hydrogel-forming copolyester can be used as a vehicle in concert with the inventive conjugates to provide the controlled release formulation aforesaid. Preferably, the hydrogel-forming copolyesters in this regard include self-solvating amphiphilic polymers or hydration-induced polymers (herein also referred to as “Gel-Former(s)” or “GF(s)”) e.g. polyethylene glycol based polymers as described in U.S. Pat. Nos. 5,714,159 and 5,612,052, incorporated herein by reference. These gel-forming polymers form a gel at or about the site of administration e.g. by injection. In an example of this embodiment, the vehicle is an absorbable gel-forming liquid made by contacting a liquid polyethylene glycol with one or more of the following cyclic monomers in the presence of a tin catalyst: glycolide, lactide, trimethylene carbonate, p-dioxanone, 1,5-dioxapan-2 dione, and ε-caprolactone. Viscosified water, pharmaceutically acceptable oils including vegetable oils such as sesame seed oil, castor oil, peanut oil and the like, and oil-based agents, polymeric agents and other non-aqueous viscous vehicles may also be employed. Examples of other vehicles include, without limitation: cellulose derivatives, polyvinylpyrrolidone, alginates, dextrans, gelatin, polyethylene glycols, polyoxyethylene ethers, polyoxypropylene ethers, and the like. Preferred cellulose derivatives include methyl cellulose, sodium carboxymethyl celluose (NaCMC) and hydroxypropyl methyl cellulose. Also contemplated as vehicles for the present invention are in situ gelling systems employing e.g. sucrose acetate isobutyrate (SAIB); poly-lactic-co-glycolic acid (PLGA); and stearic acid (SA), e.g. SA and N-methylpyrrolidone (NMP) combinations. Also, pharmaceutically acceptable aqueous compositions that optionally contain a non-ionic surfactant can also be used as vehicle in this regard.

Dosage forms other than injectable are also contemplated herein. Without limitation, the ionic conjugates of the invention can be used to make other dosage forms such as, by way of example only, oral suspensions, topical application forms, tablets, capsules and the like, including, without limitation, immediate release and controlled release forms, such as injectable formulations for intramuscular administration.

In a preferred embodiment the drug is ziprasidone and the functional polymer is formed with the monomers lactide and glycolide in a ratio of about 4:1 respectively using malic acid as an initiator (resulting in an average of 2 carboxyl groups per polymer chain). In a preferred formulation the resulting conjugate is dispersed in a polyethylene glycol based Gel Former as described above, with a drug (ziprasidone) loading in said conjugate of about 200 mgA/ml solution of conjugate in gel former; in another preferred formulation the conjugate is dispersed in sesame seed oil, the preferred drug loading being about 140 mgA/ml of ziprasidone in the form of the conjugate. In such practices, including especially the former wherein the ziprasidone conjugate is dispersed in said Gel-Former, it is preferred that the resulting injectable formulation be treated prior to administration to lower the viscosity, if needed. For example, without limitation, the resulting formulation can be subjected to mild heating, e.g. by hand or like warming, for a time sufficient prior to injection so as to facilitate complete dosing on injection, e.g. warming as aforesaid for up to about 1 hour or so.

Without limitation, the present invention can provide an injectable depot formulation for delivery of e.g. an aryl heterocyclic active agent, such as ziprasidone, at concentrations effective for treatment of illnesses such as schizophrenia over a sustained period of time, i.e. for a period of time beyond that which is obtained by immediate release injection systems. By way of example only, the present invention can provide efficacious plasma levels of active agent, e.g. ziprasidone, for at least 8 hours using typical injection volumes, e.g. about 0.1 ml to about 3 ml, about 1 ml to about 2 ml being usual. Preferably, the sustained period provided by the invention is at least 24 hours; more preferably up to about 1 week; still more preferably from about 1 week to about 2 weeks or more including up to about 8 weeks using the injection volumes aforesaid. For example, in the case of ziprasidone, the practice of the invention can deliver at least 1 to about 700 mgA, more preferably to about 350 mgA, and in one embodiment about 280 mgA, in an injection volume of about 1-2 ml for about 1 to about 2 weeks or more, including up to about 8 weeks. More preferably, about 10 to about 140 mgA for up to about 2 weeks is delivered.

The invention will for convenience now be further described using ziprasidone as the insoluble or poorly soluble pharmaceutical compound of the invention in the context of the following examples. It will be understood that the examples are illustrative and do not in any way constrain the scope of the invention. Modifications to same as appreciated by the artisan are also contemplated herein.

### EXAMPLE 1

### General Method for Preparing Carboxyl-Bearing Absorbable Polyesters, Cololyesters, and Copolyester-Carbonates

One or more cyclic monomers, namely trimethylene carbonate, I-lactide, ε-caprolactone, and glycolide, were transferred under a dry nitrogen environment into a pre-dried reactor equipped for mechanical stirring. A hydroxy acid initiator (e.g., glycolic, malic, tartaric, or citric acid) was added to the monomer mixture at a monomer/initiator molar ratio that provided the desired molecular weight; each initiator molecule resulted in one polymeric chain. The polymerization charge was heated to about 110° C. until a liquid system formed. To this was added 0.2 molar solution of stannous octanoate catalyst at a monomer/catalyst molar ratio of 5000 to 10000. The polymerization mixture was heated at 160° C. for 15 hours or until all the monomer was practically consumed (as monitored by GPC). At the conclusion of the polymerization, the polymer was heated at 110° C. under reduced pressure to remove traces of unreacted monomer. The polymer was then characterized for identity (by IR) and molecular weight (using GPC in dichloromethane). A summary of the charge, polymerization conditions, and analytical data of the resulting polymer is provided in Table I.

TABLE I
Preparation and Properties of Carboxy-bearing Polyesters,
Copolyesters, and Copolyester Carbonates

| | | | | | | Polym. | | | |
|---|---|---|---|---|---|---|---|---|---|
| | | | | Initiator^{a} | | Cond'ns: | | | |
| | Charge | | | Type, | Catalyst | Temp° C./ | GPC Data | | |
| Polymer | Monomer | Mole | Gm | M/I | M/Cat^{b} | Time (Hr) | Mₙ, Da | M_{w}, Da | PDI |
| A | Lactide | 0.4 | 57.6 | Citric | 4,500 | 160/1, | 1,680 | 2,430 | 1.45 |
| | Glycolide | 0.1 | 11.6 | Acid, 10 | | 180/10 | | | |
| B | Lactide | 0.4 | 57.6 | Citric | 4,500 | 160/1, | 1,420 | 1,950 | 1.37 |
| | Glycolide | 0.1 | 11.6 | Acid, | | 180/12 | | | |
| | | | | 7.7 | | | | | |

^{a}M/I = Molar ratio of monomer to initiator.
^{b}M/Cat = molar ratio of monomer to catalyst.
Polym. Cond'ns = polymerization conditons.
PDI = polydispersity index.

### EXAMPLE 2

### General Method for Preparing Amine-Bearing Polyester, Copolyester, and Copolyester-Carbonate

The preparation of amine-bearing polyester, copolyester, and copolyester carbonate was conducted as in Example 1 with the exception of using triethanolamine as the initiator instead of the hydroxy-carboxylic acid. The resulting polymers were characterized as noted in Example 1. Details of the polymerization charge and scheme as well as analytical data are summarized in Table II.

TABLE II
Preparation and Properties of Amine-bearing Polyesters, Copolyesters,
and Copolyester Carbonates

| | | | | | | Polym. | | | |
|---|---|---|---|---|---|---|---|---|---|
| | | | | | | Cond'ns: | | | |
| | | | | Initiator^{a} | | Temp° C./ | | | |
| | Charge | | | Type, | Catalyst | Time | GPC Data | | |
| Polymer | Monomer | Mole | Gm | M/I | M/Cat^{b} | (Hr) | Mₙ, Da | M_{w}, Da | PDI |
| C | Caprolactone | 1.247 | 142.4 | Triethanolamine, | 10,000 | 160/5 | 8,900 | 10,800 | 1.21 |
| | Glycolide | 0.066 | 7.6 | 30 | | | | | |
| D | Caprolactone | 1.247 | 142.4 | Triethanolamine, | 10,000 | 160/4 | 7,490 | 9,050 | 1.21 |
| | Glycolide | 0.066 | 7.6 | 25 | | | | | |
| E | Caprolactone | 1.247 | 142.4 | Triethanolamine, | 10,000 | 160/7 | 9,240 | 11,900 | 1.29 |
| | Glycolide | 0.066 | 7.6 | 40 | | | | | |

^{a}M/I = Molar ratio of monomer to initiator.
^{b}M/Cat = molar ratio of monomer to catalyst.
Polym. Cond'ns = polymerization conditions.
PDI = polydispersity index.

### EXAMPLE 3

### General Method for Preparing Ionic Coniugate of the Polymeric Precursors of Examples 1 and 2

A concentrated solution (20-40%) of ziprasidone in hexafluoro-isopropanol (HFIP) was mixed with a predetermined amount of concentrated solution (10-30%) of the polymer in HFIP at 25° C. The organic solvent was evaporated under reduced pressure to yield a solid or semi-solid ionic conjugate. The relative content of ionic conjugate in product was determined using differential scanning calorimetry (DSC) to compare the Tₘ and ΔH_{f} of unreacted drug to the peak temperature and area of the complex endothermic transition due to the ziprasidone/polymer ionic conjugate. The absence of the drug Tₘ signaled complete incorporation of the drug in the ionic conjugate. The conjugate formation was verified by the absence of the characteristic drug reflections in the X-ray diffraction pattern (XRD). Preparation of typical conjugate systems and their properties are summarized in Table III.

TABLE III
Preparation and Analytical Data of Conjugate Systems

| | | | DSC Data of | | | |
|---|---|---|---|---|---|---|
| | | | Conjugate Systems | | | |
| | | | Endothermic | | | |
| | | | Transition, | | | |
| | Drug/ | Polymer/ | Peak Temp, | | | |
| System | HFIP, | HFIP, | ° C./Area, J/g | | | |
| Number | gm/mL | gm/mL | 1 | 2 | 3 | XRD |
| ONE | 0.700/4 | 1.31^{c}/3 | (129-146)^{d} | — | — | amorphous |

^{c}Polymer B
^{d}Complex endotherm, could not be integrated for area.

### EXAMPLE 4

Preparation of Carboxyl-Bearing β-Cyclodextrin Derivative

Step 1: Acylation of Cyclodextrin.

Mixed acylation of β-cyclodextrin was achieved using a mixture of butyric and glutaric anhydride in the presence of p-toluene sulfonic acid as a catalyst. This was conducted as described in U.S. Pat. Nos. 5,916,883 and 6,204,256, incorporated herein by reference, to produce dried cyclodextrin butyric anhydride (CDB₃). For the particular acylated derivative relevant to this example, a glutaric/butyric/cyclodextrin weight ratio of 20.4/5.3/12.7 was used. The derivative was isolated and purified, dried, and characterized as described in U.S. Pat. Nos. 5,916,883 and 6,204,256,incorporated herein by reference.

Step 2: Grafting of CDB₃ with a Mixture of Glycolide and I-Lactide.

The grafting was conducted as described in U.S. Pat. Nos. 5,916,883 and 6,204,256. The process entailed dissolving CDB₃ (5.3 g) in a mixture of I-lactide (12.65 g) and glycolide (3.37 g) at 150° C. under a dry nitrogen environment in a predried reactor equipped for mechanical stirring. After adding a catalytic amount of stannous octanoate (57.6 μl) to the molten reactant, the polymerization was conducted at 150° C. for about 5 hours. Unreacted monomer was removed under reduced pressure at 110° C. The grafted derivative, Polymer F, was purified by precipitation of its acetone solution. The dried polymer was shown to have an equivalent weight of 618 g/Eq.

Step 3: Preparation of Ionic Conjugates of Polymer F and Ziprasidone.

The conjugates were prepared and characterized following similar protocols to those used in Example III.

TABLE IV
Preparation and Characterization
Data of Representative Conjugates

| | | | Charge | | |
|---|---|---|---|---|---|
| | | | DSC Data, | | |
| | | Zipras- | Endotherm Peak Temp, | | |
| Conjugate | Polymer | idone, | ° C., Area, J/g | | |
| Number | F, gm | gm | Endotherm 1 | Endotherm 2 | XRD |
| TWO | 1.8 | 0.2 | 138.7/12.2 | — | amorphous |
| THREE | 1.6 | 0.4 | 56.8/17.3 | 142.8/13.1 | amorphous |
| FOUR | 1.5 | 0.5 | 153.1/8.66 | 202.6/7.12 | amorphous |

### EXAMPLE 5

General Method for Preparing Liquid Gel-Forming, Controlled Release Formulation

The preparation of the formulation comprises (1) the preparation of liquid gel-forming copolyesters by end-grafting one or more cyclic monomers (e.g. dl-lactide, glycolide, caprolactone, and trimethylene carbonate) onto a liquid-polyethylene glycol (e.g. PEG-400), as described in U.S. Pat. No. 5,714,159; and (2) mechanical mixing of the solid or semi-solid conjugate (e.g. those of Example 3 and 4) at or slightly above 25° C. in the liquid gel-former.

### EXAMPLE 6

General Method for Preparing Vegetable Oil-Based, Controlled Release Formulation

The ionic conjugate (IC) was triturated using a mortar and a pestle. A pre-weighed amount of the powdered IC was transferred into a vial. Sesame oil was added into a second vial. At the time of dosing, an appropriate amount of sesame oil was withdrawn from the second vial and was added to the powdered IC. The resulting suspension was vortexed for approximately one minute to render it uniform.

### EXAMPLE 7

General Methods for Characterization of the Ionic Conjugates

1. IR spectroscopy

2. Solution NMR

3. Solid state NMR (ssNMR) CPMAS (Cross-Polarization Magic Angle Spinning) in TOSS (Total Suppression of Spinning Side Bands) Mode.

4. DSC Differential Scanning Calorimeter, samples heated from 20° C. to 250° C. at 20° C./minute.

5. X-ray diffraction (XRD).

6. Polarized light microscopy (PLM) using microscope. A small amount of sample placed on a slide glass and observed under polarized light.

7. Hot stage microscopy using microscopeand Hot Stage, A small amount of sample placed on a slide glass and observed while heating from room temperature (RT) to 230° C. at rates varying from 1 to 5° C./minute

8. Variable temperature XRD (VT-XRD), analysis performed at temperatures ranging from RT to 230° C.

9. Flow-though dissolution apparatus using distilled water in an open loop apparatus maintained at 37° C. comprising a sample holder that allows for continuous exposure to fresh water about the surface of the specimen prior to being collected in a small aliquot for analysis.

### EXAMPLE 8

### Solubility Determination of Ziprasidone from a Typical Ionic Conjugate and its Formulation in a Gel Former

The following samples were evaluated for solubility:

1. Ziprasidone-polymer ionic conjugate FIVE (40% ziprasidone, 60% polymer composed of Lactide/Glycolide/Malic acid at molar ratios of 4:1:0.65M)

2. Ziprasidone-polymer ionic conjugate in gel former (Solution of Conjugate FIVE in a typical gel former, The conjugate was dissolved in a mixture (1:1 by weight) with a mixture of two gel formers made individually of PEG-400 grafted with Trimethylene carbonate/Caprolactone/Glycolide and PEG-400 grafted with Lactide/Glycolide

3. Ziprasidone mesylate salt

4. Ziprasidone free base

Excess of each of the above sample were placed in a screw-cap vial with 2 ml of pH 7.4, PBS (Dulbecco's Phosphate-Buffered Saline) solution, and the vials were shaken continuously for 7 days. Ziprasidone free base and its mesylate salt were used as controls. The concentration of ziprasidone in solution was determined at 15 minutes, 6 hours, 24 hours, and 7 days. The HPLC samples were prepared by filtering each suspension through a 0.22-μm syringe filter without any additional dilutions

As seen from the following table, the aqueous solubility of ziprasidone is higher from both the ionic conjugate and the gel-former than ziprasidone mesylate and ziprasidone free-base as expected due to their initial amorphous nature.

TABLE V
Solubility of ionic conjugates in pH 7.4, PBS

| | | Gel-former | | | |
|---|---|---|---|---|---|
| | | Formulation of | Ionic | | |
| | | Conjugate | Conjugate | Ziprasidone | Ziprasidone |
| Characteristic | Time point | FIVE | FIVE | Mesylate | Free Base |
| Solubility (μg/ml) | 15 minutes | 0.4 μg/ml | Below | 1.5 μg/ml | Below |
| | | | detection limit | | detection limit |
| | 6 hours | 52 μg/ml | 5.4 μg/ml | 1.1 μg/ml | 0.2 μg/ml |
| | 24 hours | 42 μg/ml | 29.3 μg/ml | Not tested | 2 μg/ml |
| | 7 days | 135.3^{a} μg/ml | 6.4 μg/ml | 1.3 μg/ml | Below |
| | | | | | detection limit |

^{a}Difficulty in accurate quantitation due to polymer's interference with chromatographic conditions

What is claimed is:

1. A solid ionic conjugate comprising a pharmaceutical compound and a functional polymer, said solid ionic conjugate having aqueous solubility greater than that of said pharmaceutical compound.

2. The solid ionic conjugate of claim 1 wherein said pharmaceutical compound is insoluble or poorly soluble in water.

3. The solid ionic conjugate of claim 1 wherein said functional polymer comprises:
i) an absorbable copolyester made by ring-opening polymerization of one or more cyclic monomers selected from the group consisting of glycolide, lactide, trimethylene carbonate, p-dioxanone, 1,5-dioxapan-2-dione, and ε-caprolactone; or
ii) a carboxyl-bearing, water-insoluble cyclodextrin derivative made by a mixed partial acylation of cyclodextrin with a fatty acid anhydride and a cyclic anhydride, followed by grafting the unacylated hydroxylic group of said cyclodextrin with one or more cyclic monomers selected from glycolide, lactide, p-dioxanone, 1,5-dioxapan-2-dione, ε-caprolactone, and trimethylene carbonate.

4. The solid ionic conjugate of claim 1 wherein said pharmaceutical compound is an aryl-heterocyclic compound.

5. The solid ionic conjugate of claim 4 wherein said pharmaceutical compound is ziprasidone.

6. A pharmaceutical composition comprising the ionic conjugate of claim 1 and a pharmaceutically acceptable vehicle.

7. The pharmaceutical composition of claim 6 wherein said pharmaceutically acceptable vehicle is for controlled release or immediate release of said pharmaceutical compound.

8. The pharmaceutical composition of claim 6 wherein the functional polymer comprises:
i) an absorbable copolyester made by ring-opening polymerization of one or more of cyclic monomers selected from glycolide, lactide, trimethylene carbonate, p-dioxanone, 1,5-dioxapan-2-dione, and ε-caprolactone; or
ii) a carboxyl-bearing, water-insoluble cyclodextrin derivative made by a mixed partial acylation of cyclodextrin with a fatty acid anhydride and a cyclic anhydride, followed by grafting the unacylated hydroxylic group of said cyclodextrin with one or more of the following cyclic monomers: glycolide, lactide, p-dioxanone, 1,5-dioxapan-2-dione, ε-caprolactone, and trimethylene carbonate.

9. The pharmaceutical composition of claim 4 wherein the vehicle comprises:
i) an absorbable gel-forming liquid; or
ii) a vegetable oil.

10. The pharmaceutical composition of claim 4 wherein said pharmaceutical compound is ziprasidone; said functional polymer comprises:
i) an absorbable copolyester made by ring-opening polymerization of one or more cyclic monomers selected from glycolide, lactide, trimethylene carbonate, p-dioxanone, 1,5-dioxapan-2-dione, and ε-caprolactone; or
ii) a carboxyl-bearing, water-insoluble cyclodextrin derivative made by a mixed partial acylation of cyclodextrin with a fatty acid anhydride and a cyclic anhydride, followed by grafting the unacylated hydroxylic group of said cyclodextrin with one or more cyclic monomers selected from glycolide, lactide, p-dioxanone, 1,5-dioxapan-2-dione, ε-caprolactone, and trimethylene carbonate;
and said vehicle comprises:
i) an absorbable gel-forming liquid; or
ii) a vegetable oil.

11. A process for preparing the solid ionic conjugate of claim 1 wherein said pharmaceutical compound and a functional polymer are dissolved in an organic solvent and the ionic conjugate in substantially dry form is obtained after removing the solvent by distillation or sublimation under reduced pressure.

12. The process of claim 11 wherein said pharmaceutical compound is insoluble or poorly soluble in water.

13. The process of claim 11 wherein said pharmaceutical compound is an aryl-heterocyclic compound.

14. The process of claim 13 wherein said pharmaceutical compound is ziprasidone free base.

15. The process of claim 11 wherein said pharmaceutical compound is ziprasidone; and said functional polymer comprises:
i) an absorbable copolyester made by ring-opening polymerization of one or more cyclic monomers selected from glycolide, lactide, trimethylene carbonate, p-dioxanone, 1,5-dioxapan-2-dione, and ε-caprolactone; or
ii) a carboxyl-bearing, water-insoluble cyclodextrin derivative made by a mixed partial acylation of cyclodextrin with a fatty acid anhydride and a cyclic anhydride, followed by grafting the unacylated hydroxylic group of said cyclodextrin with one or more of the following cyclic monomers: glycolide, lactide, p-dioxanone, 1,5-dioxapan-2-dione, ε-caprolactone, and trimethylene carbonate; and said organic solvent is hexafluoro-isopropanol.
